(19) United States
(12) Patent Application Publication
(10) Pub. No.: US 2024/0081906 A1
(43) Pub. Date: Mar. 14, 2024

(54) SYSTEMS AND METHODS FOR ENERGY DELIVERY

(71) Applicant: NeuWave Medical, Inc., Madison, WI (US)

(72) Inventors: Matthew Schaning, Madison, WI (US); Nicholas Katrana, Madison, WI (US); Matthew Thiel, Madison, WI (US); Louis Mingione, Madison, WI (US); Mark Thom, Madison, WI (US); Richard Schefelker, Madison, WI (US)

(21) Appl. No.: 18/512,916

(22) Filed: Nov. 17, 2023

Related U.S. Application Data
(63) Continuation of application No. 16/297,173, filed on Mar. 8, 2019, now Pat. No. 11,832,879.

(51) Int. Cl. A61B 18/18; A61L 29/06; A61L 29/14

(52) U.S. Cl. CPC A61B 18/1815; A61L 29/06; A61L 29/14; A61B 2018/00023

(57) ABSTRACT

The present invention relates to flexible sheath assemblies capable of maintaining a desired positioning at a desired tissue region during medical procedures involving use insertion and withdrawal of medical tools through the flexible sheath, and related systems and methods.

### CROSS-REFERENCE TO RELATED APPLICATION

The present application is a continuation of U.S. patent application Ser. No. 16/297,173, filed Mar. 8, 2019, allowed as U.S. Pat. No. 11,832,879.

### FIELD OF INVENTION

The present invention relates to flexible sheath assemblies capable of maintaining a desired positioning at a desired tissue region during medical procedures involving use insertion and withdrawal of medical tools through the flexible sheath, and related systems and methods.

### BACKGROUND

Endoscopic sheaths used as guides for tool placement need to be very flexible in order to navigate through peripheral locations that may include tortuous paths, especially in bronchoscopic cases. Once at a desired location, it is difficult for the end of the sheath to remain in place as tools (e.g., obturator, flexible ablation probe, other bronchoscopic tools, etc.) are swapped and/or repositioned. Patient movements due to breathing additionally contributes to movement of the sheath.

New flexible sheaths capable of maintaining a desired positioning through use of the flexible sheaths (e.g., during medical procedures involving use of medical tools inserted and withdrawn through the sheath) are needed.

The present invention addresses this need.

### SUMMARY

The present invention relates to flexible sheath assemblies capable of maintaining a desired positioning at a desired tissue region during medical procedures involving use insertion and withdrawal of medical tools through the flexible sheath, and related systems and methods.

In certain embodiments, the present invention provides flexible sheaths for use in endoscopic procedures.

Such flexible sheaths are not limited to specific characteristics or designs.

In some embodiments, the flexible sheath comprises an elongate tubular body comprising a) an elongate tubular body proximal end having a proximal end opening and an elongate tubular body distal end having a distal end opening, and b) an elongate tubular body interior portion and an elongate tubular body exterior portion, wherein the elongate tubular body interior portion extends from the elongate tubular body proximal end to the elongate tubular body distal end, wherein the elongate tubular body exterior portion extends from the elongate tubular body proximal end to the elongate tubular body distal end; wherein the elongate tubular body interior portion comprises a hollow port extending into the proximal end opening, through the elongate tubular body proximal end, through the elongate tubular body distal end, and out the distal end opening, wherein the size of the hollow port is such that it can accommodate the passing of a properly sized tool into the hollow port, through the hollow port, and out the hollow port; wherein the diameter of the flexible sheath is less than 5 mm.

In some embodiments, the elongate tubular body interior portion further comprises an elongate tubular body coolant intake channel, an elongate tubular body distal end contained region, and an elongate tubular body coolant outtake channel, wherein the elongate tubular body distal end contained region is positioned at the elongate tubular body distal end, wherein the elongate tubular body interior portion is configured to a) receive coolant into the elongate tubular body proximal end via the elongate tubular body coolant intake channel, b) circulate the received coolant through the elongate tubular body coolant intake channel to the elongate tubular body distal end contained region, and c) circulate the coolant from the elongate tubular body distal end contained region through the elongate tubular body outtake channel and out of the elongate tubular body proximal end.

In some embodiments, the diameter of the elongate tubular body outtake channel is larger than the diameter of the elongate tubular body intake channel, or the diameter of the elongate tubular body outtake channel is smaller than the diameter of the elongate tubular body intake channel, or the diameter of the elongate tubular body outtake channel and the diameter of the elongate tubular body intake channel are identical.

In some embodiments, the hollow port, elongate tubular body intake channel, and elongate tubular body outtake channel are positioned in a multiaxial manner, or the hollow port, elongate tubular body intake channel, and elongate tubular body outtake channel are concentrically positioned in a coaxial manner.

In some embodiments, the coolant is selected from water, glycol, air, inert gasses, carbon dioxide, nitrogen, helium, sulfur hexafluoride, ionic solutions (e.g., sodium chloride with or without potassium and other ions), dextrose in water, Ringer's lactate, organic chemical solutions (e.g., ethylene glycol, diethylene glycol, or propylene glycol), oils (e.g., mineral oils, silicone oils, fluorocarbon oils), liquid metals, freons, halomethanes, liquified propane, other haloalkanes, anhydrous ammonia, sulfur dioxide, and a coolant gas compressed at or near its critical point.

In some embodiments, the distal end of the elongate tubular body exterior portion comprises one or more high thermal conductivity regions or one or more flexible-rigid regions.

In some embodiments, the one or more high thermal conductivity regions are configured to selectively attach with a tissue region upon contact with the tissue region such that the elongate tubular body distal end is stabilized in a desired position with respect to the tissue region; wherein upon attachment of the one or more high thermal conductivity regions with a tissue region, the one or more high thermal conductivity regions are configured to selectively detach from the tissue region such that the elongate tubular body distal end is destabilized from the desired position with respect to the tissue region.

In some embodiments, the one or more high thermal conductivity regions are configured to attain and maintain a temperature causing freezing of a tissue region to facilitate attachment of the tissue region to the high thermal conductivity region in order to stabilize the elongate tubular body distal end in a desired position with respect to the tissue region.

In some embodiments, temperature of the one or more high thermal conductivity regions is regulated through circulation of coolant into and out of the elongate tubular body containment region.

In some embodiments, the temperature of the one or more high thermal conductivity regions is regulated through circulation of coolant into and out of the elongate tubular body containment region via a Joule-Thompson effect, an endothermic chemical reaction, or an exothermic chemical reaction.

In some embodiments, the one or more high thermal conductivity regions comprise one or more of metal, plastic, ceramic, or mixture thereof.

In some embodiments, the one or more high thermal conductivity regions are flexible.

In some embodiments, the one or more flexible-rigid regions are configured to selectively alternate between a flexible state and a rigid state.

In some embodiments, if the one or more flexible-rigid regions are in a rigid state than the elongate tubular body distal end is stabilized in a desired position with respect to a tissue region in contact with the flexible-rigid region;

In some embodiments, if the one or more flexible-rigid regions are in a flexible state than the elongate tubular body distal end is not stabilized with respect to a tissue region in contact with the flexible-rigid region.

In some embodiments, the one or more flexible-rigid regions comprise thermoplastic polymers that have an appropriate glass-transition temperature of approximately 15-25 degrees Celsius.

In some embodiments, the thermoplastic polymer comprises copolymers of lactic acid and caprolactone.

In some embodiments, the thermoplastic polymer comprises a copolymer of L-lactide and caprolactone such as poly(L-lactide-co-caprolactone) with an L-lactide to caprolactone monomer ratio of 70:30 or less.

In some embodiments, the thermoplastic region is commercially available as PURASORB® PLC-7015 from Purac Biomaterials of Gorinchem, The Netherlands.

In some embodiments, the temperature of the one or more flexible-rigid regions is regulated through circulation of coolant into and out of the elongate tubular body containment region, wherein maintenance of a flexible-rigid region temperature at approximately −40 degrees Celsius results in a flexible state for the one or more flexible-rigid regions, wherein maintenance of a flexible-rigid region temperature at approximately −5 degrees Celsius results in a rigid state for the one or more flexible-rigid regions.

In some embodiments, the temperature of the one or more flexible-rigid regions is regulated through circulation of coolant into and out of the elongate tubular body containment region via a Joule-Thompson effect, an endothermic chemical reaction, or an exothermic chemical reaction.

In some embodiments, the properly sized tool is selected from an obturator, ablation probe, energy delivery device, biopsy tool, etc.

In some embodiments, the flexible sheath has sufficient flexibility to access a circuitous route through a subject (e.g., through a branched structure, through a bronchial tree, through any region of the body to reach a desired location).

In some embodiments, the composition of the elongate tubular body is a polymer material.

In some embodiments, the composition of the elongate tubular body is a higher temperature rated polymer material.

In some embodiments, the composition of the elongate tubular body is fluorinated ethylene propylene (FEP) or a thermoplastic copolyester (e.g., Arnitel).

In some embodiments, the composition of the elongate tubular body is a fluoropolymer. In some embodiments, the fluoropolymer is perfluoromethylalkoxy alkane (MFA) or perfluoroalkoxy alkane (PFA).

In some embodiments, the elongate tubular body interior region further comprises a steerable pull ring configured to permit a user to steer the flexible sheath in any desired manner.

In some embodiments, the flexible sheath is designed to be operational within a microwave field or microwave zone (e.g., the flexible sheaths are microwave compatible) without sustaining microwave field or microwave zone related damage.

In some embodiments, the flexible sheath is designed to be operational within a tissue region experiencing high temperatures (e.g., the flexible sheaths are thermal resistant) without sustaining high temperature related damage.

In certain embodiments, the present invention provides systems comprising a primary catheter, a flexible sheath as described above, and an energy delivery device. In some embodiments, the primary catheter is an endoscope. In some embodiments, the energy delivery device is a microwave energy delivery device.

In certain embodiments, the present invention provides methods of treating a tissue region, comprising providing a system as described above, inserting the primary catheter into a tissue region, inserting the flexible sheath through the primary catheter to a desired tissue region to be treated, securing the flexible sheath at the desired tissue region to be treated via either the one or more high thermal conductivity regions or the one or more flexible-rigid regions, inserting the energy delivery device through the flexible sheath to the desired tissue region to be treated, and treating the tissue region to be treated with the energy delivery device. In some embodiments, the tissue region to be treated is within a subject. In some embodiments, the subject is a human subject.

Additional embodiments are described herein.

### BRIEF DESCRIPTION OF THE DRAWINGS

FIGS. 1-4 show various flexible sheath embodiments.

### DETAILED DESCRIPTION

Therapeutic endoscopy or interventional endoscopy pertains to an endoscopic procedure during which a treatment (e.g., tissue ablation) (e.g., tissue collection) is carried out via the endoscope. This contrasts with diagnostic endoscopy, where the aim of the procedure is purely to visualize an internal part of a body (e.g., gastrointestinal region, respiratory region, urinary tract region, etc.) in order to aid diagnosis. In practice, a procedure which starts as a diagnostic endoscopy may become a therapeutic endoscopy depending on the findings.

Generally, therapeutic endoscopy involves the administration of an endoscope (“primary catheter”) into a body region until a natural stopping positioning is reached (e.g., until the circumference of the body region inhibits further advancement of the endoscope). Next, a flexible sheath having a circumference smaller than the circumference of the endoscope is advanced through the endoscope and to a desired body region location. Next, a therapeutic tool (e.g., an ablation energy delivery tool) (e.g., a tissue collection tool) having a circumference smaller than the diameter of the flexible sheath is advanced through the flexible sheath to the desired body region location. Next, ablation energy is delivered to the desired body region location. Upon completion of the therapeutic endoscopy, the ablation energy delivery tool is withdrawn through the flexible sheath, the flexible sheath is withdrawn through the endoscope, and the endoscope is withdrawn from the subject.

Such flexible sheaths used as guides for tool placement need to be very flexible in order to navigate through peripheral locations that may include tortuous paths, especially in bronchoscopic cases. Once at a desired location, it is difficult for the end of the flexible sheath to remain in place as tools (e.g., obturator, flexible ablation probe, other bronchoscopic tools, etc.) are swapped and/or repositioned and natural patient movement (e.g., breathing) contributes to undesired movement of the flexible sheath from the desired tissue region (e.g., the tissue region where a medical procedure is occurring).

Accordingly, new flexible sheaths capable of maintaining a desired positioning at a desired tissue region during medical procedures involving use insertion and withdrawal of medical tools are needed.

The present invention addresses this need through providing flexible sheaths capable of maintaining a desired positioning at a desired tissue region during medical procedures involving use insertion and withdrawal of medical tools and through patient body movement. Such flexible sheath assemblies are configured for use in any kind of endoscopic energy delivery procedure (e.g., tissue ablation, resection, cautery, vascular thrombosis, treatment of cardiac arrhythmias and dysrhythmias, electrosurgery, tissue harvest, etc.).

Accordingly, the present invention relates to flexible sheath assemblies capable of maintaining a desired positioning at a desired tissue region during medical procedures involving use insertion and withdrawal of medical tools through the flexible sheath, and related systems and methods.

The flexible sheaths of the present invention are not limited to particular size dimensions. Indeed, in some embodiments, the size dimension of the flexible sheath is such that it is able to fit within and pass through the lumen of a primary catheter (e.g., an endoscope). In some embodiments, the flexible sheath is of sufficient diameter (e.g. 1 mm . . . 2 mm . . . 3 mm . . . 4 mm . . . 5 mm) to accommodate within and through its interior one or more suitable tools (e.g., energy delivery device, steerable navigation catheter). In some embodiments, the flexible sheath is of sufficient length to extend from an insertion site (e.g. mouth, incision into body of subject, etc.) to a desired target region within a living body (e.g. 50 cm . . . 75 cm . . . 1 m . . . 1.5 m . . . 2 m . . . 10 m . . . 25 m, etc.). In some embodiments, the flexible sheath is of sufficient length to extend through and beyond the reach of a primary catheter (e.g., endoscope) to reach a treatment site (e.g. peripheral lung tissue, heart tissue, gastrointestinal tissue, etc.) (e.g., any desired location within a living body).

The flexible sheaths of the present invention are not limited to a particular manner of navigation through a primary catheter and/or through a body region. In some embodiments, the flexible sheath comprises a navigation and/or steering mechanism. In some embodiments, the flexible sheath is without an independent means of navigation, position recognition, or maneuvering. In some embodiments, the flexible sheath relies upon the primary catheter (e.g., endoscope) or a steerable navigation catheter for placement.

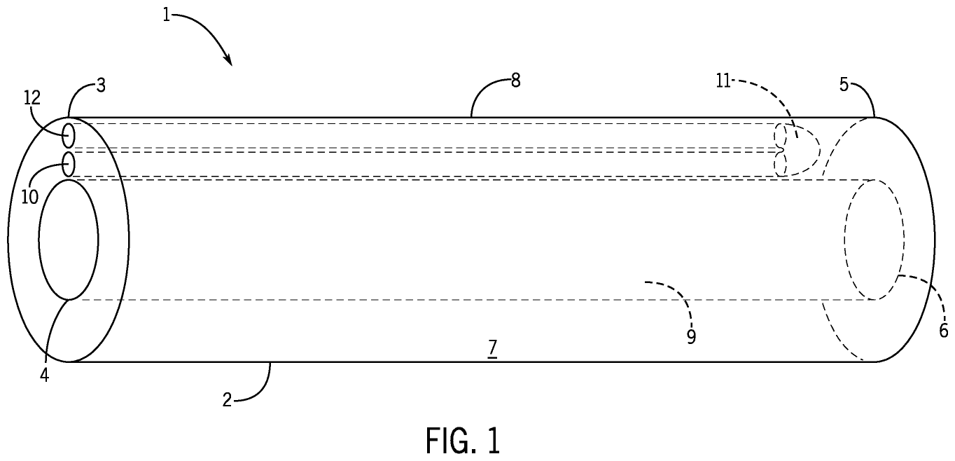

FIG. 1 shows a flexible sheath 1 embodiment of the present invention. The flexible sheath 1 is not limited to a particular design or configuration. In some embodiments, the design or configuration of the flexible sheath 1 is such that it is able to be positioned at a desired tissue region and maintain that desired positioning during medical procedures involving use insertion and withdrawal of medical tools through the flexible sheath. In some embodiments, the flexible sheath 1 has sufficient flexibility to access a circuitous route through a subject (e.g., through a branched structure, through a bronchial tree, through any region of the body to reach a desired location).

In certain embodiments, as shown in FIG. 1, the flexible sheath 1 has an elongate tubular body 2 comprising an elongate tubular body proximal end 3 having a proximal end opening 4, an elongate tubular body distal end 5 having a distal end opening 6, an elongate tubular body interior portion 7 extending from the elongate tubular body proximal end 3 to the elongate tubular body distal end 5, and an elongate tubular body exterior portion 8 extending from the elongate tubular body proximal end 3 to the elongate tubular body distal end 5. In some embodiments, the arrangement and positioning of the elongate tubular body proximal end 3, proximal end opening 4, elongate tubular body distal end 5, distal end opening 6, elongate tubular body interior portion 7, and elongate tubular body exterior portion 8 within the elongate tubular body 2 is not limited. In some embodiments, the arrangement and positioning of the elongate tubular body proximal end 3, proximal end opening 4, elongate tubular body distal end 5, distal end opening 6, elongate tubular body interior portion 7, and elongate tubular body exterior portion 8 within the elongate tubular body 2 is such that it renders the flexible sheath 1 capable of being positioned at a desired tissue region and maintaining that desired positioning during medical procedures involving use insertion and withdrawal of medical tools through the flexible sheath 1.

Still referring to FIG. 1, the elongate tubular body 2 is not limited to a particular composition. In some embodiments, the composition of the elongate tubular body 2 is any composition that renders the flexible sheath 1 capable of being positioned at a desired tissue region and maintaining that desired positioning during medical procedures involving use insertion and withdrawal of medical tools through the flexible sheath 1. In some embodiments, the composition of the elongate tubular body 2 is a polymer material. In some embodiments, the composition of the elongate tubular body 2 is a higher temperature rated polymer material. Such embodiments are not limited to a particular higher temperature rated polymer material. In some embodiments, the higher temperature rated polymer material is fluorinated ethylene propylene (FEP). In some embodiments, the higher temperature rated polymer material is a thermoplastic copolyester. In some embodiments, the thermoplastic copolyester is Arnitel. In some embodiments, the higher temperature rated polymer material is a fluoropolymer. Such embodiments are not limited to a particular fluoropolymer. In some embodiments, the fluoropolymer is perfluoromethylalkoxy alkane (MFA). In some embodiments, the fluoropolymer is perfluoroalkoxy alkane (PFA). In some embodiments, only a portion (5%, 10%, 25%, 50%, 75%, 77%, 79%, 85%, 88%, 90%, 94%, 98%, 99%, 99.999%) of the elongate tubular body 2 has a composition of a higher temperature rated polymer material. In some embodiments, only a portion (5%, 10%, 25%, 50%, 75%, 77%, 79%, 85%, 88%, 90%, 94%, 98%, 99%, 99.999%) starting from the elongate tubular body distal end 5 has a composition of a higher temperature rated polymer material. In some embodiments, the entire elongate tubular body 2 has a composition of a higher temperature rated polymer material.

Still referring to FIG. 1, the flexible sheath 1 is configured such that devices (e.g., medical devices) can be inserted and withdrawn through the elongate tubular body interior portion 7. Examples of such devices that can be inserted and withdrawn through the elongate tubular body interior portion 7 include, but are not limited to, an obturator, ablation probe, energy delivery device, biopsy tool, etc.

Still referring to FIG. 1, the elongate tubular body interior portion 7 is not limited to particular configuration permitting the insertion and withdrawal of devices. In some embodiments, the elongate tubular body interior portion 7 has therein a hollow port 9 extending from the proximal end opening 4, through the elongate tubular body proximal end 3, through the elongate tubular body distal end 5, and out the distal end opening 6. The hollow port 9 is not limited to a particular size. In some embodiments, the size of the hollow port 9 is such that it can accommodate the insertion and withdrawal of a properly sized device (e.g., a device having a circumference smaller than the circumference of the hollow port 9) through its entirety. In some embodiments, the size of the hollow port 9 is such that it can accommodate the insertion and withdrawal of a properly sized device (e.g., a device having a circumference smaller than the circumference of the hollow port 9) through its entirety without compromising the ability of the flexible sheath 1 to be positioned at a desired tissue region and maintaining that desired positioning during medical procedures.

Still referring to FIG. 1, the elongate tubular body interior portion 7 is configured to circulate a coolant for purposes of maintaining the distal end of the elongate tubular body distal end 5 at a desired temperature. The elongate tubular body interior portion 7 is not limited to a particular manner of circulating a coolant for purposes of maintaining the distal end of the elongate tubular body distal end 5 at a desired temperature. In some embodiments, as shown in FIG. 1, the elongate tubular body interior portion 7 is able to circulate a coolant for purposes of maintaining the distal end of the elongate tubular body distal end 5 at a desired temperature through use of an elongate tubular body coolant intake channel 10, an elongate tubular body distal end contained region 11 positioned at the elongate tubular body distal end 5, and an elongate tubular body coolant outtake channel 12. Indeed, in some embodiments, the elongate tubular body interior portion 7 is configured to a) receive coolant into the elongate tubular body proximal end 3 via the elongate tubular body coolant intake channel 10, b) circulate the received coolant through the elongate tubular body coolant intake channel 10 to the elongate tubular body distal end contained region 11, and c) circulate the coolant from the elongate tubular body distal end contained region 11 through the elongate tubular body outtake channel 12 and out of the elongate tubular body proximal end 3.

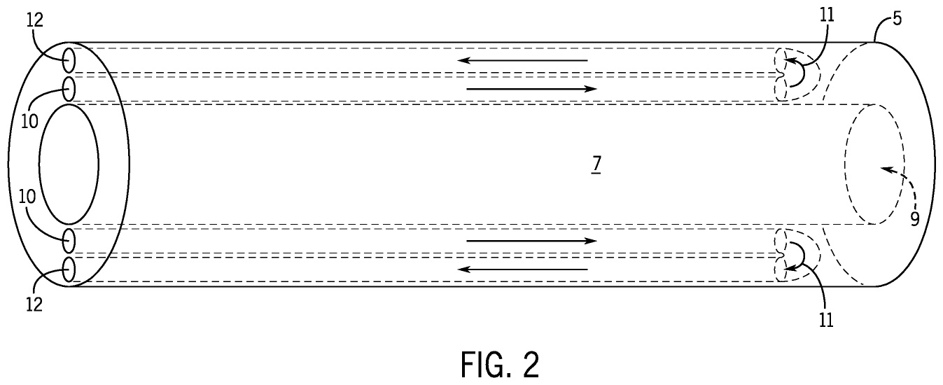

The hollow tube 9, elongate tubular body coolant intake channel 10, elongate tubular body distal end contained region 11, and elongate tubular body coolant outtake channel 12 are not limited to a particular positioning within the elongate tubular body interior portion 7. In some embodiments, as shown in FIG. 1, the hollow tube 9, elongate tubular body coolant intake channel 10, elongate tubular body distal end contained region 11, and elongate tubular body coolant outtake channel 12 are positioned in a multiaxial relationship. In some embodiments, as shown in FIG. 2, the hollow tube 9, elongate tubular body coolant intake channel 10, elongate tubular body distal end contained region 11, and elongate tubular body coolant outtake channel 12 are positioned in a concentric relationship with, for example, the hollow tube 9 surrounded by the elongate tubular body coolant outtake channel 12 which is surrounded by the elongate tubular body coolant intake channel 10, and the elongate tubular body distal end contained region 11 positioned at the distal end of the elongate tubular body exterior 8.

The elongate tubular body coolant intake channel 10 and elongate tubular body coolant outtake channel 12 are not limited to particular sizes. In some embodiments, the diameter of the elongate tubular body outtake channel 12 is larger than the diameter of the elongate tubular body intake channel 10. In some embodiments, the diameter of the elongate tubular body outtake channel 12 is smaller than the diameter of the elongate tubular body intake channel 10. In some embodiments, the diameter of the elongate tubular body outtake channel 12 and the diameter of the elongate tubular body intake channel are identical 10.

The elongate tubular body distal end contained region 11 is not limited to a particular size. In some embodiments, the size of the elongate tubular body distal end contained region 11 is such that it is able to accommodate coolant circulated through the elongate tubular body coolant intake channel 10 for purposes of maintaining the distal end of the elongate tubular body distal end 5 at a desired temperature (described in more detail below).

Such embodiments are not limited to use of a specific type or kind of coolant. In some embodiments, the coolant is selected from water, glycol, air, inert gasses, carbon dioxide, nitrogen, helium, sulfur hexafluoride, ionic solutions (e.g., sodium chloride with or without potassium and other ions), dextrose in water, Ringer's lactate, organic chemical solutions (e.g., ethylene glycol, diethylene glycol, or propylene glycol), oils (e.g., mineral oils, silicone oils, fluorocarbon oils), liquid metals, freons, halomethanes, liquified propane, other haloalkanes, anhydrous ammonia, sulfur dioxide, and a coolant gas compressed at or near its critical point.

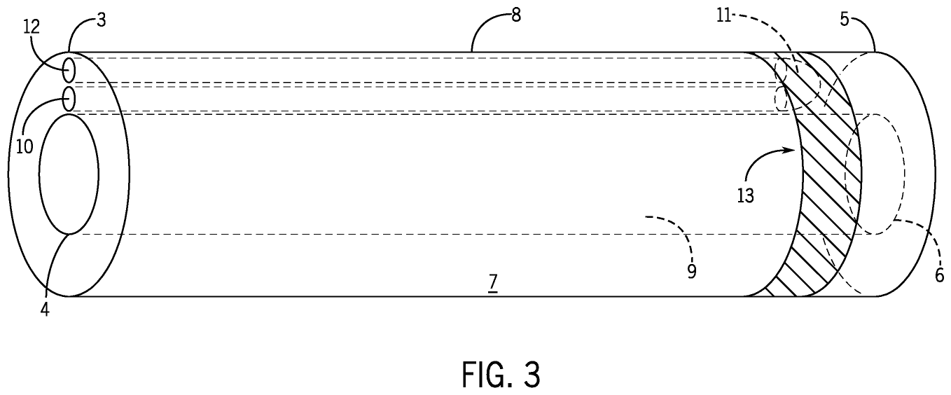

FIG. 3 shows a flexible sheath 1 wherein the distal end of the elongate tubular body exterior portion 5 comprises one or more high thermal conductivity regions 13 (as shown in FIG. 3 there is one high thermal conductivity region 13). In some embodiments, the high thermal conductivity region 13 is configured to selectively attach with a tissue region upon contact with the tissue region such that the elongate tubular body distal end 5 is stabilized in a desired position with respect to the tissue region.

The high thermal conductivity region 13 is not limited to a particular positioning along the distal end of the elongate tubular body exterior portion 8. In some embodiments, as shown in FIG. 3, the high thermal conductivity region 13 wraps around a portion (e.g., 1%, 5%, 10%, 25%, 45%, 49.9%, 50%, 55%, 62%, 70%, 79.5%, 85%, 90%, 92%, 93.5%, 98%, 99%, 99.99%) of the distal end of the elongate tubular body exterior portion 8. In some embodiments, the high thermal conductivity region 13 covers only a portion (e.g., 1%, 5%, 10%, 25%, 45%, 49.9%, 50%, 55%, 62%, 70%, 79.5%, 85%, 90%, 92%, 93.5%, 98%, 99%, 99.99%) of the of the distal end of the elongate tubular body exterior portion 8 but does not wrap around the entirety of the distal end of the elongate tubular body exterior portion 8.

The flexible sheath is not limited to a particular number of high thermal conductivity regions 13. In some embodiments, there is one high thermal conductivity region 13, as shown in FIG. 3. In some embodiments, there are 2, 3, 5, 8, 10, 25, 100, 1000, etc. high thermal conductivity regions 13 positioned along the distal end of the elongate tubular body exterior portion 8.

The high thermal conductivity region 13 is not limited to a particular manner of selectively attaching with a tissue region upon contact with the tissue region such that the elongate tubular body distal end 5 is stabilized in a desired position with respect to the tissue region. In some embodiments, the high thermal conductivity region 13 is able to selectively attach with a tissue region upon contact with the tissue region such that the elongate tubular body distal end 5 is stabilized in a desired position with respect to the tissue region through selective temperature adjustment of the high thermal conductivity region 13. For example, in some embodiments, upon positioning of the flexible sheath 1 to a desired location, selective reduction of the high thermal conductivity region 13 permits the tissue region to freeze onto the high thermal conductivity region 13 thereby securing the elongate tubular body distal end 5 to that desired location. In some embodiments, selective reduction of the high thermal conductivity region 13 permits the tissue region to freeze onto the high thermal conductivity region 13 thereby securing the elongate tubular body distal end 5 to that desired location during insertion and withdrawal of the devices through the hollow port 9. In some embodiments wherein the high thermal conductivity region 13 is secured with a tissue region, selectively increasing the temperature of the high thermal conductivity region 13 results in detachment (e.g., un-freezing) of the tissue region from the high thermal conductivity region 13.

The flexible sheath 1 is not limited to particular manner of selectively maintaining the temperature of the high thermal conductivity region 13. In some embodiments, the temperature of the high thermal conductivity region 13 is maintained and/or adjusted through circulation coolant into and out of the elongate tubular body distal end contained region 11. Indeed, the positioning of the high thermal conductivity region 13 is such that coolant circulated into the elongate tubular body distal end contained region 11 results in temperature adjustment/maintenance for the high thermal conductivity region 13. For example, in some embodiments, upon positioning of the flexible sheath 1 at a desired tissue location, coolant is circulated into the elongate tubular body distal end contained region 11 such that the temperature of the high thermal conductivity region 13 is lowered to a level wherein the tissue region attaches (e.g., freezes) onto the high thermal conductivity region. For example, maintaining tissue region attachment with the high thermal conductivity region 13 is accomplished through maintaining sufficient circulation of coolant into the elongate tubular body distal end contained region 11 to retain the high thermal conductivity region 13 at a temperature permitting such attachment. For example, increasing the temperature of the high thermal conductivity region 13 through reducing circulation of coolant into the elongate tubular body distal end contained region 11 facilitates detachment of the tissue region from the high thermal conductivity region 13. For example, increasing the temperature of the high thermal conductivity region 13 through increasing circulation of a higher temperature coolant into the elongate tubular body distal end contained region 11 facilitates detachment of the tissue region from the high thermal conductivity region 13.

In some embodiments, the temperature of the high thermal conductivity region 13 is regulated through circulation of coolant into and out of the elongate tubular body containment region 11 via a Joule-Thompson effect, an endothermic chemical reaction, or an exothermic chemical reaction.

In some embodiments, the material of the high thermal conductivity region 13 comprises one or more of metal, plastic, ceramic, or mixture thereof. In some embodiments, the material of the high thermal conductivity region 13 is flexible (e.g., as flexible as the remainder of the flexible sheath 1).

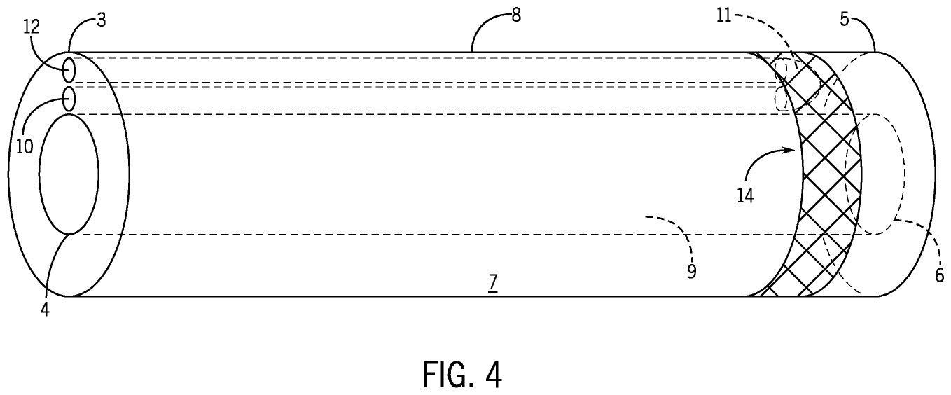

FIG. 4 shows a flexible sheath 1 wherein the distal end of the elongate tubular body exterior portion 5 comprises a flexible-rigid region 14. In some embodiments, the flexible-rigid region 14 is configured to selectively alternate between a flexible state and a rigid state. In some embodiments, the flexible-rigid region 14 is configured to selectively alternate between a flexible state and a rigid state for purposes of stabilizing the elongate tubular body distal end 5 with respect to a tissue region in contact with the flexible-rigid region 14.

The flexible-rigid region 14 is not limited to a particular positioning along the distal end of the elongate tubular body exterior portion 8. In some embodiments, as shown in FIG. 4, the flexible-rigid region 14 wraps around a portion (e.g., 1%, 5%, 10%, 25%, 45%, 49.9%, 50%, 55%, 62%, 70%, 79.5%, 85%, 90%, 92%, 93.5%, 98%, 99%, 99.99%) of the distal end of the elongate tubular body exterior portion 8. In some embodiments, the flexible-rigid region 14 covers only a portion (e.g., 1%, 5%, 10%, 25%, 45%, 49.9%, 50%, 55%, 62%, 70%, 79.5%, 85%, 90%, 92%, 93.5%, 98%, 99%, 99.99%) of the of the distal end of the elongate tubular body exterior portion 8 but does not wrap around the entirety of the distal end of the elongate tubular body exterior portion 8.

The flexible-rigid region 14 is not limited to a particular manner of selectively stabilizing the elongate tubular body distal end 5 with respect to a tissue region in contact with the flexible-rigid region 14. In some embodiments, flexible-rigid region 14 is able to selectively stabilize the elongate tubular body distal end 5 with respect to a tissue region in contact with the flexible-rigid region 14 through selective temperature adjustment of the flexible-rigid region 14. For example, in some embodiments, upon positioning of the flexible sheath 1 to a desired location, selectively increasing the temperature of the flexible-rigid region 14 renders the flexible-rigid region 14 into a rigid state which facilitates an anchoring of the elongate tubular body distal end 5 at the desired location. For example, in some embodiments, upon positioning of the flexible sheath 1 to a desired location, selectively increasing the temperature of the flexible-rigid region 14 renders the flexible-rigid region 14 into a rigid state which facilitates an anchoring of the elongate tubular body distal end 5 at the desired location during insertion and withdrawal of the devices through the hollow port 9. In some embodiments wherein the flexible-rigid region 14 is anchored at a tissue region (e.g., the flexible-rigid region 14 is in a rigid state), selectively decreasing the temperature of the flexible-rigid region 14 results in transition of the flexible-rigid region 14 to a flexible state which thereby releases the elongate tubular body distal end 5 from an anchoring with the tissue region.

The flexible sheath 1 is not limited to particular manner of selectively maintaining the temperature of the flexible-rigid region 14 for purposes of alternating between a flexible or rigid state. In some embodiments, the temperature of the flexible-rigid region 14 is maintained and/or adjusted through circulation coolant into and out of the elongate tubular body distal end contained region 11. Indeed, the positioning of the flexible-rigid region 14 is such that coolant circulated into the elongate tubular body distal end contained region 11 results in temperature adjustment/maintenance for the flexible-rigid region 14. For example, in some embodiments, upon positioning of the flexible sheath 1 at a desired tissue location, coolant is circulated into the elongate tubular body distal end contained region 11 such that the temperature of the flexible-rigid region 14 is either increased or decreased or maintained to achieve a desired rigidity or flexibility for the flexible-rigid region 14 thereby achieving either an anchoring with a tissue region or de-anchoring with the tissue region.

In some embodiments, the flexible-rigid region 14 comprises thermoplastic polymers that have an appropriate glass-transition temperature of approximately 15-25 degrees Celsius. In some embodiments, the thermoplastic polymer comprises copolymers of lactic acid and caprolactone. In some embodiments, the thermoplastic polymer comprises a copolymer of L-lactide and caprolactone such as poly(L-lactide-co-caprolactone) with an L-lactide to caprolactone monomer ratio of 70:30 or less. In some embodiments, the thermoplastic region is commercially available as PURASORB® PLC-7015 from Purac Biomaterials of Gorinchem, The Netherlands.

In some embodiments, the temperature of the flexible-rigid region 14 is regulated through circulation of coolant into and out of the elongate tubular body containment region 11 via a Joule-Thompson effect, an endothermic chemical reaction, or an exothermic chemical reaction.

In some embodiments, the flexible sheaths further contain a steerable pull ring. Such embodiments are not limited to a particular configuration for the steerable pull ring. In some embodiments, the steerable pull ring has any configuration that permits a user to manually steer the flexible sheath via manipulation of the steerable pull ring (e.g., manipulation of one or both of the wires results in a curving or steering of the sheath).

In some embodiments, the steerable pull ring permits the flexible sheath to be steered in any desired manner or direction. For example, in some embodiments, the steerable pull ring permits the flexible sheath to be steered at any desired curve angle (e.g., from 1 to 180 degrees). In some embodiments, the steerable pull ring permits the flexible sheath to be steered at any desired bend angle (e.g., from 1 to 360 degrees). In some embodiments, the steerable pull ring permits the flexible sheath to be steered at any desired bend radius (e.g., from 1 to 360 degrees). In some embodiments, the steerable pull ring permits the flexible sheath to be steered at any desired curve diameter. In some embodiments, the steerable pull ring permits the flexible sheath to be steered at any desired reach (e.g., from 0.1 to 100 mm). In some embodiments, the steerable pull ring permits the flexible sheath to be steered at any desired curl. In some embodiments, the steerable pull ring permits the flexible sheath to be steered at any desired sweep. In some embodiments, the steerable pull ring permits the flexible sheath to be steered at any desired curve (e.g., symmetrical or asymmetrical) (e.g., multi-curve or compound curve). In some embodiments, the steerable pull ring permits the flexible sheath to be steered at any desired loop. In some embodiments, the steerable pull ring permits the flexible sheath to be steered at any desired deflection (e.g., on-plane deflection, off plane deflection).

In some embodiments, the present invention provides systems for therapeutic endoscopic procedures wherein flexible sheaths as described herein, primary catheters, and one or more suitable tools (e.g., energy delivery device, steerable navigation catheter) are provided.

Such embodiments are not limited to a particular type or kind of primary catheter. In some embodiments, the present invention primary catheter is an endoscope. In some embodiments, any suitable endoscope known to those in the art finds use as a primary catheter in the present invention. In some embodiments, a primary catheter adopts characteristics of one or more endoscopes and/or bronchoscopes known in the art, as well as characteristics described herein. One type of conventional flexible bronchoscope is described in U.S. Pat. No. 4,880,015, herein incorporated by reference in its entirety. The bronchoscope measures 790 mm in length and has two main parts, a working head and an insertion tube. The working head contains an eyepiece; an ocular lens with a diopter adjusting ring; attachments for suction tubing, a suction valve, and light source; and an access port or biopsy inlet, through which various devices and fluids can be passed into the working channel and out the distal end of the bronchoscope. The working head is attached to the insertion tube, which typically measures 580 mm in length and 6.3 mm in diameter. The insertion tube contains fiberoptic bundles, which terminate in the objective lens at the distal tip, light guides, and a working channel. Other endoscopes and bronchoscopes which may find use in embodiments of the present invention, or portions of which may find use with the present invention, are described in U.S. Pat. Nos. 7,473,219; 6,086,529; 4,586,491; 7,263,997; 7,233,820; and 6,174,307.

Such embodiments are not limited to a particular type or kind of steerable navigation catheter. In some embodiments, a steerable navigation catheter is configured to fit within the lumen of a primary catheter (e.g., endoscope) and a flexible sheath. In some embodiments, a steerable navigation catheter is of sufficient length to extend from an insertion site (e.g. mouth, incision into body of subject, etc.) to a treatment site (e.g. 50 cm . . . 75 cm . . . 1 m . . . 1.5 m . . . 2 m . . . 5 m . . . 15 m). In some embodiments, a channel catheter is of sufficient length to extend beyond the reach of a primary catheter (e.g., endoscope) to reach a treatment site (e.g. peripheral lung tissue). In some embodiments, a steerable navigation catheter engages a flexible sheath such that movement of the steerable navigation catheter results in synchronous movement of the flexible sheath. In some embodiments, as a steerable navigation catheter is inserted along a path in a subject, the flexible sheath surrounding the steerable navigation catheter moves with it. In some embodiments, a flexible sheath is placed within a subject by a steerable navigation catheter. In some embodiments, a steerable navigation catheter can be disengaged from a flexible sheath. In some embodiments, disengagement of a steerable navigation catheter and flexible sheath allows movement of the steerable navigation catheter further along a pathway without movement of the flexible sheath. In some embodiments, disengagement of a steerable navigation catheter and flexible sheath allows retraction of the steerable navigation catheter through the flexible sheath without movement of the flexible sheath.

Such embodiments are not limited to a particular type or kind of energy delivery device (e.g., ablation device, surgical device, etc.) (see, e.g., U.S. Pat. Nos. 7,101,369, 7,033,352, 6,893,436, 6,878,147, 6,823,218, 6,817,999, 6,635,055, 6,471,696, 6,383,182, 6,312,427, 6,287,302, 6,277,113, 6,251,128, 6,245,062, 6,026,331, 6,016,811, 5,810,803, 5,800,494, 5,788,692, 5,405,346, 4,494,539, U.S. patent application Ser. Nos. 11/728,460, 11/728,457, 11/728,428, 11/237,136, 11/236,985, 10/980,699, 10/961,994, 10/961,761, 10/834,802, 10/370,179, 09/847,181; Great Britain Patent Application Nos. 2,406,521, 2,388,039; European Patent No. 1395190; and International Patent Application Nos. WO 06/008481, WO 06/002943, WO 05/034783, WO 04/112628, WO 04/033039, WO 04/026122, WO 03/088858, WO 03/039385 WO 95/04385; each herein incorporated by reference in their entireties). Such energy delivery devices are not limited to emitting a particular kind of energy. In some embodiments, the energy delivery devices are capable of emitting radiofrequency energy. In some embodiments, the energy delivery devices are capable of emitting microwave energy. Such devices include any and all medical, veterinary, and research applications devices configured for energy emission, as well as devices used in agricultural settings, manufacturing settings, mechanical settings, or any other application where energy is to be delivered.

The systems for therapeutic endoscopic procedures of the present invention are not limited to particular uses. Indeed, such systems of the present invention are designed for use in any setting wherein the emission of energy is applicable. Such uses include any and all medical, veterinary, and research applications. In addition, the systems and devices of the present invention may be used in agricultural settings, manufacturing settings, mechanical settings, or any other application where energy is to be delivered.

In some embodiments, the systems are configured for any type of procedure wherein the flexible sheath described herein can find use. For example, the systems find use for open surgery, percutaneous, intravascular, intracardiac, intraluminal, endoscopic, laparoscopic, or surgical delivery of energy.

The present invention is not limited by the nature of the target tissue or region. Uses include, but are not limited to, treatment of heart arrhythmia, tumor ablation (benign and malignant), control of bleeding during surgery, after trauma, for any other control of bleeding, removal of soft tissue, tissue resection and harvest, treatment of varicose veins, intraluminal tissue ablation (e.g., to treat esophageal pathologies such as Barrett's Esophagus and esophageal adenocarcinoma), treatment of bony tumors, normal bone, and benign bony conditions, intraocular uses, uses in cosmetic surgery, treatment of pathologies of the central nervous system including brain tumors and electrical disturbances, sterilization procedures (e.g., ablation of the fallopian tubes) and cauterization of blood vessels or tissue for any purposes. In some embodiments, the surgical application comprises ablation therapy (e.g., to achieve coagulative necrosis). In some embodiments, the surgical application comprises tumor ablation to target, for example, metastatic tumors. In some embodiments, the systems including the flexible sheath described herein are configured for movement and positioning, with minimal damage to the tissue or organism, at any desired location, including but not limited to, the lungs, brain, neck, chest, abdomen, and pelvis. In some embodiments, the systems are configured for guided delivery, for example, by computerized tomography, ultrasound, magnetic resonance imaging, fluoroscopy, and the like. Indeed, in some embodiments, all inserted components of such a system are configured for movement along a narrow and circuitous path through a subject (e.g. through a branched structure, through the bronchial tree, etc.).

In certain embodiments, the present invention provides methods of treating a tissue region, comprising providing a tissue region and a system described herein (e.g., a primary catheter (e.g., an endoscope), a flexible sheath as described herein, and an energy delivery device (e.g., a microwave ablation catheter), and at least one of the following components: a processor, a power supply, a temperature monitor, an imager, a tuning system, a temperature reduction system, and/or a device placement system); positioning a portion of the energy delivery device in the vicinity of the tissue region, and delivering an amount of energy with the device to the tissue region. In some embodiments, the tissue region is a tumor. In some embodiments, the delivering of the energy results in, for example, the ablation of the tissue region and/or thrombosis of a blood vessel, and/or electroporation of a tissue region. In some embodiments, the tissue region is a tumor. In some embodiments, the tissue region comprises one or more of the lung, heart, liver, genitalia, stomach, lung, large intestine, small intestine, brain, neck, bone, kidney, muscle, tendon, blood vessel, prostate, bladder, and spinal cord.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

We claim:

1. A flexible sheath for use in endoscopic procedures, comprising an elongate tubular body; the elongate tubular body comprising
a) an elongate tubular body proximal end having a proximal end opening and an elongate tubular body distal end having a distal end opening, and an elongate tubular main body region positioned between the elongate tubular body proximal end and the elongate tubular body distal end;
b) an elongate tubular body interior portion and an elongate tubular body exterior portion, wherein the elongate tubular body interior portion extends from the elongate tubular body proximal end to the elongate tubular body distal end, wherein the elongate tubular body exterior portion extends from the elongate tubular body proximal end to the elongate tubular body distal end; and
c) a flexible-rigid region positioned only in the elongate tubular body distal end;
wherein if the flexible-rigid region is in a rigid state then the elongate tubular body distal end is stabilized in a desired position with respect to a tissue region in contact with the flexible-rigid region; and
wherein if the flexible-rigid region is in a flexible state then the elongate tubular body distal end is not stabilized with respect to a tissue region in contact with the flexible-rigid region.

2. The flexible sheath of claim 1, wherein the flexible-rigid region is positioned on a most exterior region of the elongate tubular body exterior portion.

3. The flexible sheath of claim 1,
wherein the elongate tubular body interior portion comprises a hollow port extending into the proximal end opening, through the elongate tubular body proximal end, through the elongate tubular body distal end, and out the distal end opening, wherein the size of the hollow port is such that it can accommodate the passing of a properly sized tool into the hollow port, through the hollow port, and out the hollow port;

4. The flexible sheath of claim 1, wherein the diameter of the flexible sheath is less than 5 mm.

5. The flexible sheath of claim 1, wherein the flexible-rigid region is configured to selectively alternate between a flexible state and a rigid state.

6. The flexible sheath of claim 1,
wherein the elongate tubular body interior portion further comprises an elongate tubular body coolant intake channel, an elongate tubular body distal end contained region, and an elongate tubular body coolant outtake channel,
wherein the elongate tubular body distal end contained region is positioned at the elongate tubular body distal end,
wherein the elongate tubular body interior portion is configured to a) receive coolant into the elongate tubular body proximal end via the elongate tubular body coolant intake channel, b) circulate the received coolant through the elongate tubular body coolant intake channel to the elongate tubular body distal end contained region, and c) circulate the coolant from the elongate tubular body distal end contained region through the elongate tubular body outtake channel and out of the elongate tubular body proximal end,
wherein
the diameter of the elongate tubular body outtake channel is larger than the diameter of the elongate tubular body intake channel, or
the diameter of the elongate tubular body outtake channel is smaller than the diameter of the elongate tubular body intake channel, or
the diameter of the elongate tubular body outtake channel and the diameter of the elongate tubular body intake channel are identical,
wherein
the hollow port, elongate tubular body intake channel, and elongate tubular body outtake channel are positioned in a multiaxial manner, or
the hollow port, elongate tubular body intake channel, and elongate tubular body outtake channel are concentrically positioned in a coaxial manner,
wherein the coolant is selected from water, glycol, air, inert gasses, carbon dioxide, nitrogen, helium, sulfur hexafluoride, ionic solutions, dextrose in water, Ringer's lactate, organic chemical solutions, oils, liquid metals, freons, halomethanes, liquified propane, other haloalkanes, anhydrous ammonia, sulfur dioxide, and a coolant gas compressed at or near its critical point.

7. The flexible sheath of claim 6, wherein the one or more flexible-rigid regions comprise thermoplastic polymers that have an appropriate glass-transition temperature of 15-25 degrees Celsius.

8. The flexible sheath of claim 7,
wherein the thermoplastic polymer comprises copolymers of lactic acid and caprolactone,
wherein the thermoplastic polymer comprises a copolymer of L-lactide and caprolactone with an L-lactide to caprolactone monomer ratio of 70:30 or less.

9. The flexible sheath of claim 6,
wherein the temperature of the flexible-rigid region is regulated through circulation of coolant into and out of the elongate tubular body containment region,
wherein maintenance of a flexible-rigid region temperature at approximately −40 degrees Celsius results in a flexible state for the flexible-rigid region,
wherein maintenance of a flexible-rigid region temperature at approximately −5 degrees Celsius results in a rigid state for the flexible-rigid region,
wherein the temperature of the flexible-rigid region is regulated through circulation of coolant into and out of the elongate tubular body containment region via a Joule-Thompson effect, an endothermic chemical reaction, or an exothermic chemical reaction,
wherein the elongate tubular body interior region further comprises a steerable pull ring configured to permit a user to steer the flexible sheath in any desired manner,
wherein the flexible sheath is designed to be operational within a microwave field or microwave zone without sustaining microwave field or microwave zone related damage,
wherein the flexible sheath is designed to be operational within a tissue region experiencing high temperatures without sustaining high temperature related damage.

10. The flexible sheath of claim 3,
wherein the properly sized tool is selected from an obturator, ablation probe, energy delivery device, or biopsy tool,
wherein the flexible sheath has sufficient flexibility to access a circuitous route through a subject,
wherein the composition of the elongate tubular body is a polymer material,
wherein the composition of the elongate tubular body is selected from a higher temperature rated polymer material, fluorinated ethylene propylene (PEP), a thermoplastic copolyester, and a fluoropolymer or perfluoroalkoxy alkane (PFA).

11. A flexible sheath for use in endoscopic procedures, comprising an elongate tubular body; the elongate tubular body comprising
a) an elongate tubular body proximal end having a proximal end opening and an elongate tubular body distal end having a distal end opening, and an elongate tubular main body region positioned between the elongate tubular body proximal end and the elongate tubular body distal end;
b) an elongate tubular body interior portion and an elongate tubular body exterior portion, wherein the elongate tubular body interior portion extends from the elongate tubular body proximal end to the elongate tubular body distal end, wherein the elongate tubular body exterior portion extends from the elongate tubular body proximal end to the elongate tubular body distal end; and
c) a flexible-rigid region positioned on the most exterior region of the elongate tubular body exterior portion;
wherein if the flexible-rigid region is in a rigid state then the elongate tubular body distal end is stabilized in a desired position with respect to a tissue region in contact with the flexible-rigid region; and
wherein if the flexible-rigid region is in a flexible state then the elongate tubular body distal end is not stabilized with respect to a tissue region in contact with the flexible-rigid region.

12. The flexible sheath of claim 11, wherein the flexible-rigid region is positioned only in the elongate tubular body distal end.

13. The flexible sheath of claim 11,
wherein the elongate tubular body interior portion comprises a hollow port extending into the proximal end opening, through the elongate tubular body proximal end, through the elongate tubular body distal end, and out the distal end opening, wherein the size of the hollow port is such that it can accommodate the passing of a properly sized tool into the hollow port, through the hollow port, and out the hollow port;

14. The flexible sheath of claim 11, wherein the diameter of the flexible sheath is less than 5 mm.

15. The flexible sheath of claim 11, wherein the flexible-rigid region is configured to selectively alternate between a flexible state and a rigid state.

16. The flexible sheath of claim 11,
wherein the elongate tubular body interior portion further comprises an elongate tubular body coolant intake channel, an elongate tubular body distal end contained region, and an elongate tubular body coolant outtake channel,
wherein the elongate tubular body distal end contained region is positioned at the elongate tubular body distal end,
wherein the elongate tubular body interior portion is configured to a) receive coolant into the elongate tubular body proximal end via the elongate tubular body coolant intake channel, b) circulate the received coolant through the elongate tubular body coolant intake channel to the elongate tubular body distal end contained region, and c) circulate the coolant from the elongate tubular body distal end contained region through the elongate tubular body outtake channel and out of the elongate tubular body proximal end,
wherein
the diameter of the elongate tubular body outtake channel is larger than the diameter of the elongate tubular body intake channel, or
the diameter of the elongate tubular body outtake channel is smaller than the diameter of the elongate tubular body intake channel, or
the diameter of the elongate tubular body outtake channel and the diameter of the elongate tubular body intake channel are identical,
wherein
the hollow port, elongate tubular body intake channel, and elongate tubular body outtake channel are positioned in a multiaxial manner, or
the hollow port, elongate tubular body intake channel, and elongate tubular body outtake channel are concentrically positioned in a coaxial manner,
wherein the coolant is selected from water, glycol, air, inert gasses, carbon dioxide, nitrogen, helium, sulfur hexafluoride, ionic solutions, dextrose in water, Ringer's lactate, organic chemical solutions, oils, liquid metals, freons, halomethanes, liquified propane, other haloalkanes, anhydrous ammonia, sulfur dioxide, and a coolant gas compressed at or near its critical point.

17. The flexible sheath of claim 16, wherein the one or more flexible-rigid regions comprise thermoplastic polymers that have an appropriate glass-transition temperature of 15-25 degrees Celsius.

18. The flexible sheath of claim 17,
wherein the thermoplastic polymer comprises copolymers of lactic acid and caprolactone,
wherein the thermoplastic polymer comprises a copolymer of L-lactide and caprolactone with an L-lactide to caprolactone monomer ratio of 70:30 or less.

19. The flexible sheath of claim 16,
wherein the temperature of the flexible-rigid region is regulated through circulation of coolant into and out of the elongate tubular body containment region,
wherein maintenance of a flexible-rigid region temperature at approximately −40 degrees Celsius results in a flexible state for the flexible-rigid region,
wherein maintenance of a flexible-rigid region temperature at approximately −5 degrees Celsius results in a rigid state for the flexible-rigid region,
wherein the temperature of the flexible-rigid region is regulated through circulation of coolant into and out of the elongate tubular body containment region via a Joule-Thompson effect, an endothermic chemical reaction, or an exothermic chemical reaction,
wherein the elongate tubular body interior region further comprises a steerable pull ring configured to permit a user to steer the flexible sheath in any desired manner,
wherein the flexible sheath is designed to be operational within a microwave field or microwave zone without sustaining microwave field or microwave zone related damage,
wherein the flexible sheath is designed to be operational within a tissue region experiencing high temperatures without sustaining high temperature related damage.

20. The flexible sheath of claim 13,
wherein the properly sized tool is selected from an obturator, ablation probe, energy delivery device, or biopsy tool,
wherein the flexible sheath has sufficient flexibility to access a circuitous route through a subject,
wherein the composition of the elongate tubular body is a polymer material, wherein the composition of the elongate tubular body is selected from a higher temperature rated polymer material, fluorinated ethylene propylene (PEP), a thermoplastic copolyester, and a fluoropolymer or perfluoroalkoxy alkane (PFA).
